(19) United States
(12) Patent Application Publication
(10) Pub. No.: US 2011/0236977 A1
(43) Pub. Date: Sep. 29, 2011

(54) DENTAL STEM CELL DIFFERENTIATION

(75) Inventors: Jeremy J. Mao, Closter, NJ (US); Rujing Yang, Zhengzhou (CN); Chang Hun Lee, New York, NY (US); Sarah Kennedy, Brooklyn, NY (US)

(73) Assignee: The Trustees of Columbia University in the City of New York, New York, NY (US)

(21) Appl. No.: 12/936,383

(22) PCT Filed: Apr. 2, 2009
(86) PCT No.: PCT/US09/39360

§ 371 Date: May 25, 2011

Related U.S. Application Data
(62) Division of application No. 61/041,686, filed on Apr. 2, 2008.

(51) Int. Cl. C12N 5/0775; C12N 5/071; C12N 15/85

(52) U.S. Cl. 435/455; 435/377; 435/366; 435/325

(57) ABSTRACT

Provided is a method of preparing an embryonic stem cell-like cell, a method of preparing an insulin-secreting cell or pancreatic beta-like cell, a method of preparing a chondrocyte-like cell, a method of preparing a myocyte-like cell, and a method of preparing a hair follicle-like cell. A composition comprising a dental stem cell and an insulin-secreting cell or a pancreatic beta-like cell is also provided. Further, a composition comprising (a) a dental stem cell and (b) a chondrocyte-like cell, a myocyte-like cell, or a hair follicle-like cell is provided. Additionally provided is an insulin-secreting cell or a pancreatic beta-like cell differentiated from a dental stem cell. Further provided is a chondrocyte-like cell, a myocyte-like cell, or a hair follicle-like cell, derived from a dental stem cell.

### CROSS-REFERENCE TO RELATED APPLICATIONS

This application claims the benefit of U.S. Provisional Application No. 61/041,686, filed Apr. 2, 2008, which is incorporated herein by reference in its entirety.

### STATEMENT REGARDING FEDERALLY SPONSORED RESEARCH OR DEVELOPMENT

This invention was made with government support under Grants No. R01DE15391 and R01EB005256 awarded by The National Institutes of Health. The government has certain rights in the invention.

### BACKGROUND

The present application generally relates to stem cell differentiation. More specifically, the invention is directed to differentiation of dental stem cells into pancreatic islet beta cells, myoblasts, chondrocytes, and hair follicle cells, and the dedifferentiation of dental stem cells in a pluripotent/totipotent embryonic stem cell-like state.

Stem cells have become the centerpiece of regenerative medicine (Alhadlaq and Mao, 2004; Marion and Mao, 2006). Different types of stem cells include embryonic stem cells, amniotic fluid stem cells, umbilical cord stem cells, and adult stem cells from bone marrow, skeletal muscle and adipose tissue (Mao et al., 2007).

The tooth functions to process food, and also is important for aesthetics, vocal communicating, including speech in humans, and digestion. Tooth pulp is neural crest-derived mesenchymal tissue, and its genesis relies on epithelial-mesenchymal interactions. Dental-pulp stem/progenitor cells, or “dental stem cells” (DSCs) express the embryonic stem cell markers Nanog and Oct4, suggesting their primitive status. These cells from the tooth can differentiate into osteoblasts, neuron-like cells and adipocytes (Miura et al., 2003; U.S. Patent Publication US20070274958A1). See also PCT patent publications WO04073633A2, WO03066840A2, WO07014639A2, WO06010600A2, WO0207679A2.

Insulin-producing cells (IPCs) have been derived from embryonic stem cells and postnatal stem cells isolated from anatomic structures such as amniotic fluid, bone marrow, and adipose tissue (D'Amour et al, 2006; Lumelsky et al., 2001). A common challenge for this task is insulin yield.

There is a need for further development of applications for dental stem cells, including methods and media to direct their differentiation into a broader range of tissues, for example insulin-producing cells, or to direct dedifferentiation into a more undifferentiated state. The present application addresses that need.

### SUMMARY

This invention is based in part on the discovery that dental stem cells can differentiate into insulin-secreting cells or pancreatic beta-like cells, chondrocyte-like cells, myocyte-like cells and hair follicle-like cells when cultured in the right media. Media that effect differentiation of dental stem cells into the above cells has also been identified. Also identified herein is methods and media for preparing an embryonic stem cell-like cell derived from a dental stem cell.

In some embodiments, the invention is directed to a method of preparing an embryonic stem cell-like cell. The method comprises culturing a dental stem cell in a medium that maintains an embryonic stem cell, under conditions such that the dental stem cell dedifferentiates into the embryonic stem cell-like cell.

In other embodiments, the invention is directed to a method of preparing an insulin-secreting cell. The method comprises incubating a dental stem cell in a medium that induces the differentiation of a dental stem cell into an insulin-secreting cell, under conditions such that the dental stem cell differentiates into the insulin-secreting cell.

In additional embodiments, the invention is directed to a method of preparing a chondrocyte-like cell. The method comprises incubating a dental stem cell in a medium that induces the differentiation of a dental stem cell into a chondrocyte-like cell, under conditions such that the dental stem cell differentiates into the chondrocyte-like cell.

The invention is also directed to a method of preparing a myocyte-like cell. The method comprises incubating a dental stem cell in a medium that induces the differentiation of a dental stem cell into a myocyte-like cell, under conditions such that the dental stem cell differentiates into the myocyte-like cell.

Additionally, the invention is directed to a method of preparing a hair follicle-like cell. The method comprises incubating a dental stem cell in a medium that induces the differentiation of a dental stem cell into a hair follicle-like cell, under conditions such that the dental stem cell differentiates into the hair follicle-like cell.

In further embodiments, the invention is directed to a composition comprising a dental stem cell and an insulin-secreting cell.

In additional embodiments, the invention is directed to a composition of (a) a dental stem cell and (b) a chondrocyte-like cell, a myocyte-like cell, or a hair follicle-like cell.

The invention is also directed to an insulin-secreting cell differentiated from a dental stem cell.

Further, the invention is directed to a chondrocyte-like cell, a myocyte-like cell, or a hair follicle-like cell, derived from a dental stem cell.

In further embodiments, the invention is directed to a composition comprising (a) a dental stem cell and (b) a chondrocyte-like cell, a myocyte-like cell, or a hair follicle-like cell.

The invention is additionally directed to a pancreatic beta-like cell differentiated from a dental stem cell.

In other embodiments, the invention is directed to a chondrocyte-like cell, a myocyte-like cell, or a hair follicle-like cell, derived from a dental stem cell.

### BRIEF DESCRIPTION OF THE DRAWINGS

FIG. 1 is micrographs showing differentiation of dental stem cells (DSCs) into pancreatic beta cells. Undifferentiated DSCs in DMEM show clusters of cells (A, B). In comparison, differentiated DSCs show drastically different cell morphology as a cluster (C, D).

FIG. 2 is micrographs of cells immunostained for insulin or PDX1, showing the differentiation of dental stem cells (DSCs) into pancreatic beta-like cells. DSCs started to differentiate 1 wk after disassociation from cell clusters. Treated DSCs (A) show positive expression of insulin (B), a key secretory molecule of native pancreatic beta cells, and PDX1 (C), a beta cell transcriptional factor. In comparison, control DSCs failed to express either insulin or PDX1 (D, E, F).

FIG. 3 is micrographs of cells immunostained for C-peptide, showing differentiation of dental stem cells (DSCs) into pancreatic beta-like cells. Images showing in 1 wk differentiation after disassociation from cell clusters. Treated DSCs show positive expression of C-peptide (A,B), a molecule expressed by native pancreatic beta cells. In comparison, control DSCs failed to express C-peptide (C and D).

FIG. 4 is a graph of the quantification of insulin in cells by ELISA, showing differentiation of dental stem cells (DSCs) into pancreatic beta cells. There was significantly increased medium insulin content in cultures of DSC-derived pancreatic beta cells than in control cells. Thus, DSC-derived pancreatic beta cells not only were positive to immunostaining with insulin, PDX1 and C-peptide, but also produce more insulin than controls.

FIG. 5 is micrographs of chondrogenic differentiation of dental stem cells (DSCs) two weeks after induction of differentiation. Safranin O (Saf-O) stains glycosaminoglycans that are one of the primary extracellular matrix molecules in cartilage and synthesized by chondrocytes (B, D). Hematoxylin and eosin stain (H&E) staining shows that pellets formed by DSCs contain somewhat homogenously distributed cells (A, C).

FIG. 6 is micrographs showing differentiation of dental stem cells (“TSCs”=tooth-derived stem cells) into hair follicle cells. The TSCs differentiated into dermal papilla cells and outer root sheath cells of the hair follicle. TSCs differentiated into dermal papilla (DP) cells express Lef1, a transcriptional factor expressed by native dermal papilla cells (b), in comparison with TSCs without DP differentiation (a). TSCs also differentiated into outer root sheath cells (ORS) by expressing CD59, a transcriptional factor expressed by native ORS cells (d), in comparison with TSCs without ORS differentiation (c).

FIG. 7 is micrographs and a graph showing myogenic differentiation of dental stem cells (TSC). After 1 wk differentiation, TSCs expressed myoD (b), a transcriptional factor expressed by native myoblasts, in comparison with reduced myoD expression in control cells (a). These data are quantified in e. By 4 wks, desmin expression was marked as shown in c, with an overlay of DAPI stained cell nuclei in d.

FIG. 8 is fluorescent micrographs showing the immunostaining of proinsulin and Pdx-1 of dental-pulp stem/progenitor cells.

FIG. 9 is a graph showing insulin secretion of MSC- and TSC-derived insulin-producing cells (IPCs).

FIG. 10 is a photograph, micrographs and graphs showing characteristics of DSCs including expressed markers.

FIG. 11 is micrographs showing characteristics, including expressed markers, of DSCs when cultured in particular media.

### DETAILED DESCRIPTION

The present invention is based in part on the discovery that dental stem cells can differentiate into insulin-secreting cells or pancreatic beta-like cells, chondrocyte-like cells, myocyte-like cells and hair follicle-like cells when cultured in the right media. Media that effect differentiation of dental stem cells into the above cells has also been identified, as has media that causes dental stem cells to dedifferentiate into an embryonic stem cell-like cell.

In some embodiments, the invention is directed to a method of preparing an insulin-secreting cell or a pancreatic beta-like cell. The method comprises incubating a dental stem cell in a medium that induces the differentiation of a dental stem cell into an insulin-secreting cell, under conditions such that the dental stem cell differentiates into the insulin-secreting cell.

As used herein, a stem cell is a relatively undifferentiated cell capable of self-renewal through mitotic cell division and also capable of differentiating into more specialized cell types. As is known in the art, stem cells include embryonic stem cells, which are totipotent, i.e., capable of differentiating into all cell types of the organism from which they were derived, and adult stem cells, which are pluripotent (capable of differentiating into almost all cell types including types from all three germ layers), multipotent (capable of differentiating into several cell types of a closely related family of cells), or unipotent (capable of differentiating into only one type of cell but distinguished from non-stem cells by the ability to self-renew by mitosis).

As used herein, a dental stem cell (DSC; also known as tooth-derived stem cell=TSC) is a stem cell derived from vertebrate tooth pulp. They can be from any tooth of any vertebrate that has teeth. In some embodiments, the dental stem cell is derived from a deciduous tooth. In other embodiments, the dental stem cell is derived from a premolar, a molar, an incisor or a canine DSCs have previously been shown to be capable of differentiating into neuron-like cells, osteoblasts and adipocytes. Since those tissues are derived from the mesoderm and ectoderm germ layers, DSCs evidently have the capacity to differentiate into cells of two different germ layers. As evidenced by the data provided in the examples below, DSCs are also unexpectedly capable of differentiating into pancreatic beta cell-like cells, which are derived from the endoderm germ layer. Thus, DSCs are capable of differentiating into cells of all three germ types, and thus are pluripotent cells. Because most adult stem cells are not capable of differentiating into cells from all three germ layers, the finding herein that DSCs have that capability is unexpected.

As used herein, an insulin-secreting cell is a cell that produces insulin. A pancreatic beta-like cell is a cell derived from a stem cell that produces insulin and PDX-1, and/or C-peptide, which are markers characteristic of pancreatic beta cells. These cells can be used for treatment of type 1 diabetes.

The dental stem cell in these embodiments can be from any species. In some embodiments, the dental stem cell is a mammalian cell, for example a human cell, a rat cell, a rabbit cell, or a mouse cell.

Any medium known to differentiate a stem cell into an insulin-producing cell or a pancreatic beta-like cell can be used in these methods. See, e.g., Examples 1 and 2. In some embodiments, the medium for these methods comprises activin, exendin, pentagastrin, hepatocyte growth factor, and/or noggin. In certain specific embodiments, the medium comprises 0.001-1000 nM activin A, 0.001-1000 nM extendin-4 and 0.001-1000 nM pentagastrin. In more specific embodiments, the medium comprises 0.5-10 nM activin-A, 2-30 nM exendin-4, 2-30 nM pentagastrin and 20-300 pM. In additional embodiments, the medium comprises low glucose DMEM supplemented with about 10 mM nicotinamide, about 2 nM activin-A, about 10 nM exendin-4, about 100 pM hepatocyte growth factor, about 10 nM pentagastrin, B-27 supplement, N-2 Supplement, and at least one antibiotic. Where the medium comprises noggin, that compound is generally added to a concentration of about 100-1000 ng/ml, more specifically about 400 ng/ml.

In some embodiments, the method further comprises testing the cell for a characteristic of a pancreatic beta cell. Any such characteristic can be tested in this method. In some embodiments, the characteristic is the secretion of insulin. Another characteristic that can be tested is the production of PDX1. A further characteristic that can be tested is the production of C-peptide. These characteristics can be tested by any means known in the art. In some embodiments, they are tested by ELISA or fluorescent antibody cell staining. In further embodiments the cells are tested for all three characteristics.

In other embodiments, the invention is directed to a method of preparing a chondrocyte-like cell. The method comprises incubating a dental stem cell in a medium that induces the differentiation of a dental stem cell into a chondrocyte-like cell, under conditions such that the dental stem cell differentiates into the chondrocyte-like cell.

As used herein, a chondrocyte-like cell is a cell derived from a stem cell that stains with safranin O, and/or comprises glycosaminoglycans. Chondrocyte-like cells can be used for the treatment of arthritis or for augmentative or reconstructive surgery.

Any medium known to differentiate stem cells into chondrocyte-like cells can be used in these methods. See, e.g., Example 1. In some embodiments, the medium comprises TGF-β3.

In various embodiments, the method further comprises testing the cell for a characteristic of a chondrocyte. Any characteristic that distinguishes a chondrocyte from other cells can be tested. In some embodiments, the cell is tested for safranin O staining and/or glycosaminoglycan content.

The invention is also directed to a method of preparing a myocyte-like cell. The method comprises incubating a dental stem cell in a medium that induces the differentiation of a dental stem cell into a myocyte-like cell, under conditions such that the dental stem cell differentiates into the myocyte-like cell.

As used herein, a myocyte-like cell is a cell derived from a stem cell that comprises myoD, myf5, desmin and/or myosin. Myocyte-like cells can be used to treat muscular dystrophy, atrophy, or for the enhancement of muscle strength.

Any medium that induces the differentiation of a stem cell into a myocyte-like cell can be used for these methods. In some embodiments, the medium comprises dexamethasone and hydrocortisone.

In some embodiments, the method further comprises testing the cell for a characteristic of a myocyte. Any characteristic that distinguishes a myocyte from other cells can be tested. In some embodiments, the cell is tested for myoD, myf5, desmin and/or myosin, by any means known in the art.

Additionally, the invention is directed to a method of preparing a hair follicle-like cell. The method comprises incubating a dental stem cell in a medium that induces the differentiation of a dental stem cell into a hair follicle-like cell, under conditions such that the dental stem cell differentiates into the hair follicle-like cell. Any medium that induces the differentiation of a stem cell into a hair follicle-like cell can be used in these methods. In some embodiments, the medium is dermal papilla media or outer root sheath media.

As used herein, a hair follicle-like cell is a cell derived from a stem cell that exhibits a characteristic of a hair follicle. Examples of such characteristics are the presence of CD44, Lef1, CD59 and/or CK14. Hair follicle-like cells can be used for hair follicle regeneration in the treatment of alopecia or baldness.

In various embodiments, these methods further comprise testing the cell for a characteristic of a hair follicle. Any characteristic that distinguishes a hair follicle cell from other cells can be tested, by any means known in the art. Examples include CD44, Lef1, CD59 and/or CK14.

It has also been discovered that dental stem cells can be dedifferentiated into embryonic stem cell-like cells. As such, the dedifferentiated cells are pluripotent or totipotent. The invention is thus also directed to a method of preparing an embryonic stem cell-like cell. The method comprises culturing a dental stem cell in a medium that maintains an embryonic stem cell, under conditions such that the dental stem cell dedifferentiates into the embryonic stem cell-like cell. Any medium known to be useful for maintaining stem cells in their undifferentiated state can be used for these methods. In some embodiments (as in Example 3), the medium comprises leukemia inhibitory factor (LIF) and Knockout™ Serum Replacement. Additionally, in various embodiments, the medium comprises feeder cells, such as irradiated mouse embryonic fibroblasts, as in Example 3.

The dedifferentiation of the dental stem cells into embryonic stem cell-like cells can be monitored by any method known, for example by testing the cells for markers indicative of undifferentiated or pluripotent or totipotent cells, for example alkaline phosphatase, Oct-3/4, Nanog and SSEA4.

In various embodiments, these methods further comprise growing the embryonic stem cell-like cell in a second medium that causes the cell to differentiate, for example into an insulin-secreting cell, a chondrocyte cell, a myocyte cell, or a hair follicle-like cell.

In some embodiments of any of the above methods, the cell is transfected with a nucleic acid encoding a protein or a functional polynucleotide that is expressed by the cell. The cell may be transfected either before or after the differentiation of the dental stem cell into the specialized cell (i.e., insulin-producing, pancreatic beta-like, chondrocyte-like, myocyte-like, or hair follicle-like cell) or the dedifferentiation of the dental stem cell into the embryonic stem cell-like cell.

In some aspects of these embodiments, the nucleic acid encodes a protein, for example a therapeutic protein, such as: a protein missing in the intended recipient of the cell, e.g., a clotting factor, common gamma chain (γ_{c}), or adenosine deaminase; a structural protein, e.g., collagen; an antigen of a disease organism to induce immunity; a growth factor, e.g., to promote the differentiation of the cell (such as transfecting the cell with proinsulin or hepatocyte growth factor to promote production of insulin or differentiation into a pancreatic beta-like cell, or TGF-β3 to promote differentiation into a chondrocyte-like cell); or a protein that provides therapy for a growth factor deficiency (e.g., IL-12) or to fight cancer or infection (e.g., γ-interferon).

In other aspects the nucleic acid encodes a functional polynucleotide. As used herein, a functional polynucleotide is a polynucleotide that has a known function, for example an miRNA, an aptamer, or an antisense RNA. The functional polynucleotides can promote the differentiation of the cells (as in, e.g., Nakajima et al., 2006) or can be utilized for any other purpose, for example, as a cancer therapy (as in, e.g., Saito et al., 2006).

The invention is additionally directed to a composition comprising a dental stem cell and an insulin-secreting cell or a pancreatic beta-like cell. Such a composition would only be expected to occur when a culture of dental stem cells are differentiating into an insulin-secreting cell or a pancreatic beta-like cell. In some embodiments, this composition is in any of the above-identified media that can induce differentiation of a stem cell into an insulin-secreting cell or a pancreatic beta-like cell.

The application is further directed to a composition comprising (a) a dental stem cell and (b) a chondrocyte-like cell, a myocyte-like cell, or a hair follicle-like cell. These compositions would only be expected to occur when a culture of dental stem cells are differentiating into chondrocyte-like cells, myocyte-like cells, or hair follicle-like cells.

Additionally, the application is directed to an insulin-secreting cell or a pancreatic beta-like cell differentiated from a dental stem cell. Similarly, the application is also directed to a chondrocyte-like cell, a myocyte-like cell, or a hair follicle-like cell, derived from a dental stem cell.

Preferred embodiments are described in the following examples. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the example.

### Example 1

### Differentiation of Dental Stem Cells into Pancreatic Beta Cells, Myocytes, Hair Follicle Cells and Chondrocytes

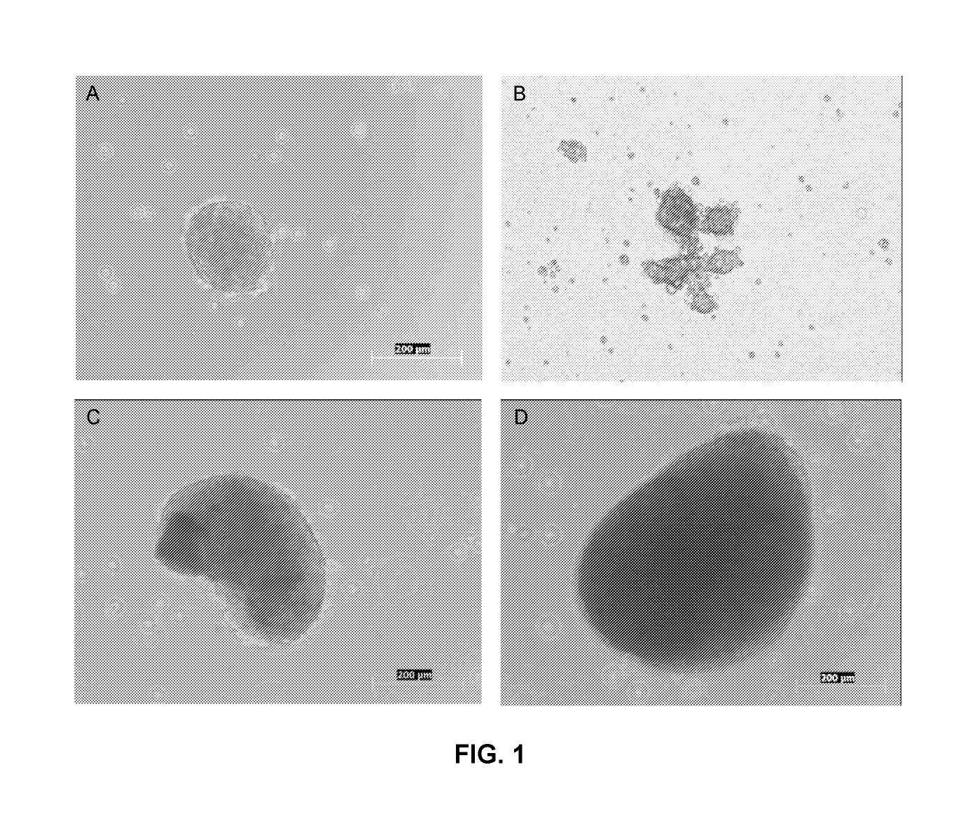

Deciduous teeth were extracted under sterile conditions from healthy children with an age range of 5-7 years in the Pediatric Dentistry Clinic of Columbia University Medical Center, under IRB approval and following informed consent procedures. The extracted deciduous teeth were transported under sterile conditions to the research laboratory and immediately processed. The pulp tissue of the deciduous teeth was removed by mechanical passaging and further digested with collagenase (Alhadlaq and Mao, 2004; Marion and Mao, 2006). The isolated mononucleated cells (FIGS. 1A, B) showed rapid proliferation rates in comparison with bone marrow derived mesenchymal stem cells (MSC) and adipose stem cells (ASC) as described by Alhadlaq and Mao (2004) and Marion and Mao (2006).

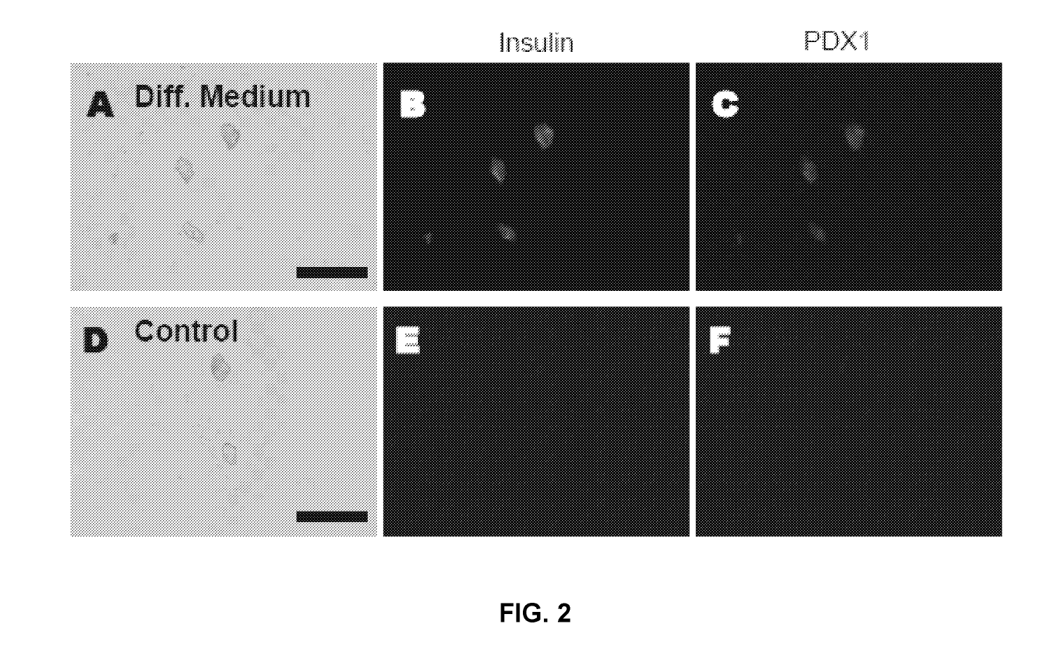

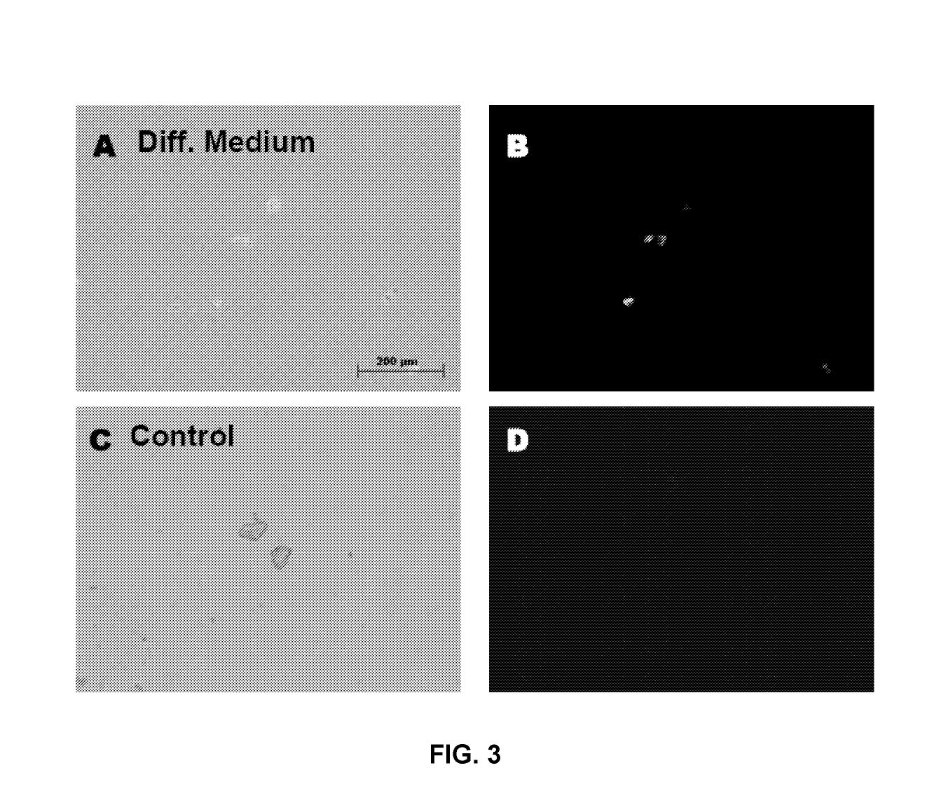

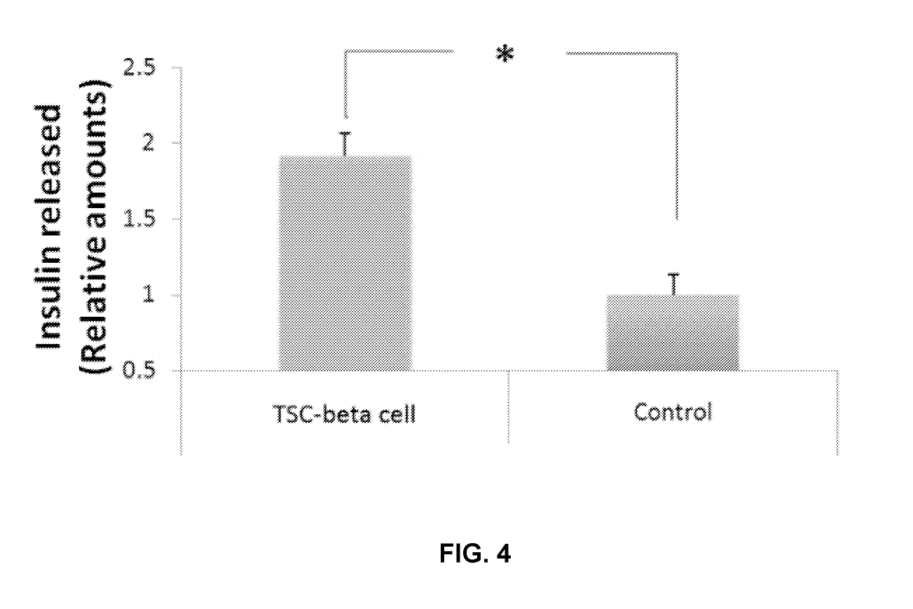

For differentiation into beta cells, 300,000 cells/mL of DSCs were plated in ultra-low attachment 6-well plate (Corning) and cultured for 3 days. The cells were then cultured in differentiation medium, which is low glucose DMEM supplemented with 10 mM nicotinamide, 2 nM activin-A 2, 10 nM exendin-4, 100 pM hepatocyte growth factor, 10 nM pentagastrin, B-27 serum-free supplement, N-2 Supplement, 1% antibiotics, was applied with fresh medium change on every third day. At 4 wks, the isolated cells formed multiple clusters and had proliferated at a remarkable rate (FIGS. 1C, D). At this point, cells were trypsinized to obtain single cells and then re-plated in tissue culture for 24 hrs. Immunoreactivities for insulin, PDX1, and C-peptide were then assayed (FIGS. 2, 3). ELISA was performed to measure the insulin content of DSC-derived pancreatic beta cells (FIG. 4).

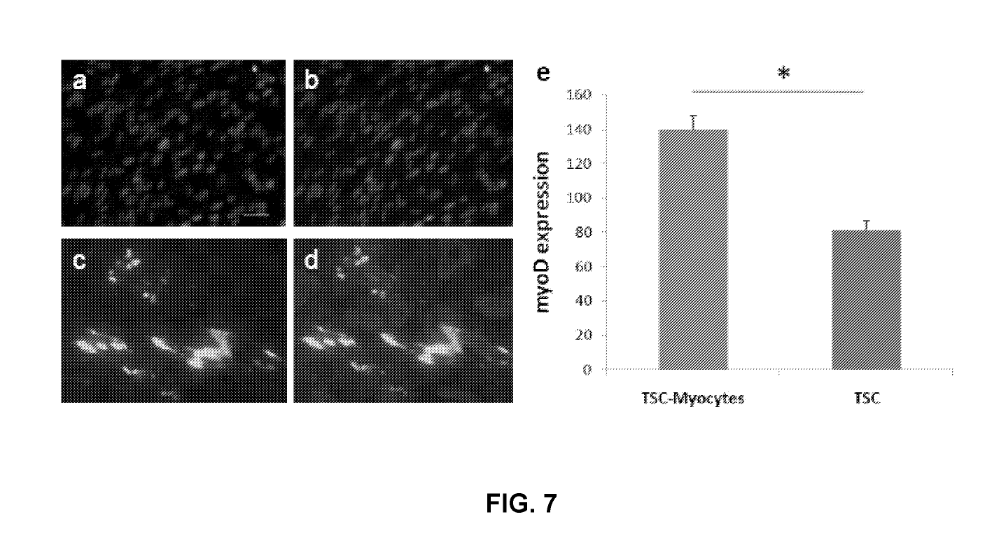

Dental stem cells (DSCs) were culture-expanded in monolayer in 24-well tissue culture plates at a density of 2000/cm² in DMEM supplemented with 10% FBS, 1% Antibiotic, with fresh medium change every 3-4 days. At ˜80% confluence, DSCs were differentiated using two differentiation medium cocktails. Cocktail A consisted of DMEM+10% FBS+1% antibiotic +5% horse serum +0.1 μM dexamethasone+50 μM hydrocortisone. Cocktail B consisted of DMEM+1% Antibiotic +5% horse serum. By 2 and 4 wks of the treatment with Cocktails A and B, the expression of transcription factors myoD and myf5 (2 wks), desmin, a myocyte structural protein and myosin (4 wks), were assayed using immunohistochemistry (FIG. 7). The expression of myoD, myf5, desmin, and myosin was also quantified using an infrared imaging system (Odyssey®; LI-COR, Lincoln, Nebr.) using Alexa Fluor® 680 (Invitrogen, Carlsbad, Calif.) and IRDye® 800CW (LI-COR, Lincoln, Nebr.), as secondary antibodies conjugated with infrared fluoropores. Data were analyzed using two samples Student t-test and p<0.05 was considered significant (FIG. 7e).

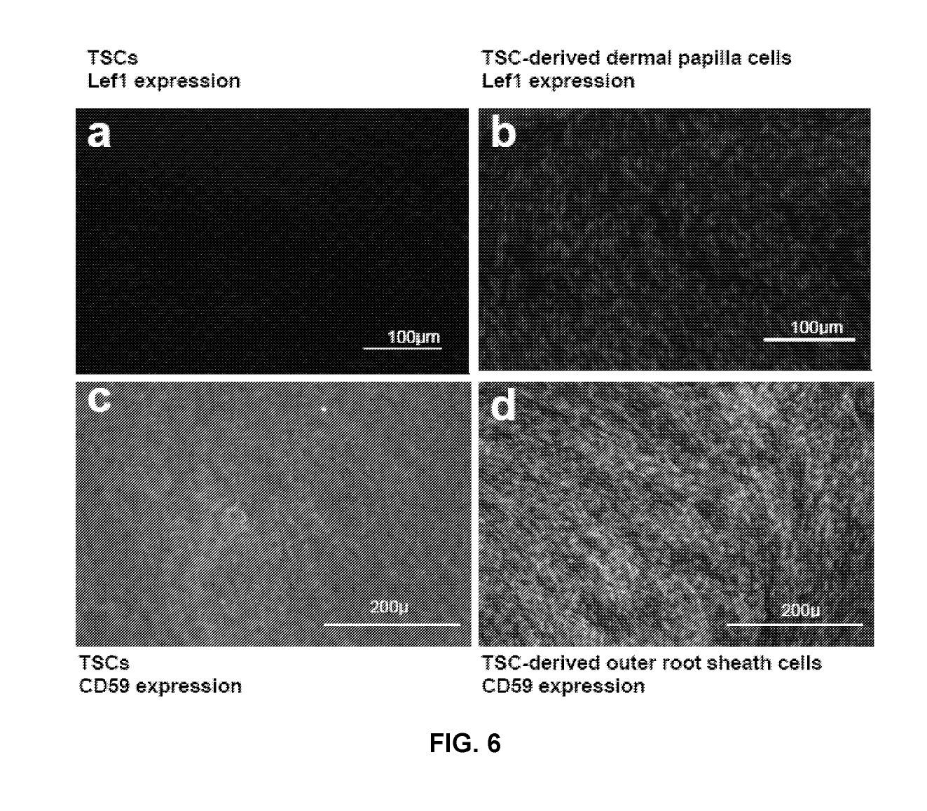

DSCs were seeded in 6-well plates with 50,000 cells per well and incubated overnight in either 2 mL of dermal papilla (DP) media or outer root sheath (ORS) media (Celprogen, San Pedro, Calif.) at 5% CO₂ and 95% humidity. After DSCs attached overnight, transwells with a pore size of 0.4 μm were placed into the wells of seeded DSCs with each of 50,000 DP and ORS cells. After 7 and 14 days, the transwells were removed. Immunostaining was applied to detect the presence of DP markers CD44 and Lef1, and ORS markers CD59 and CK14 in DSCs, using primary antibodies and AlexaFluor secondary antibodies (FIG. 6).

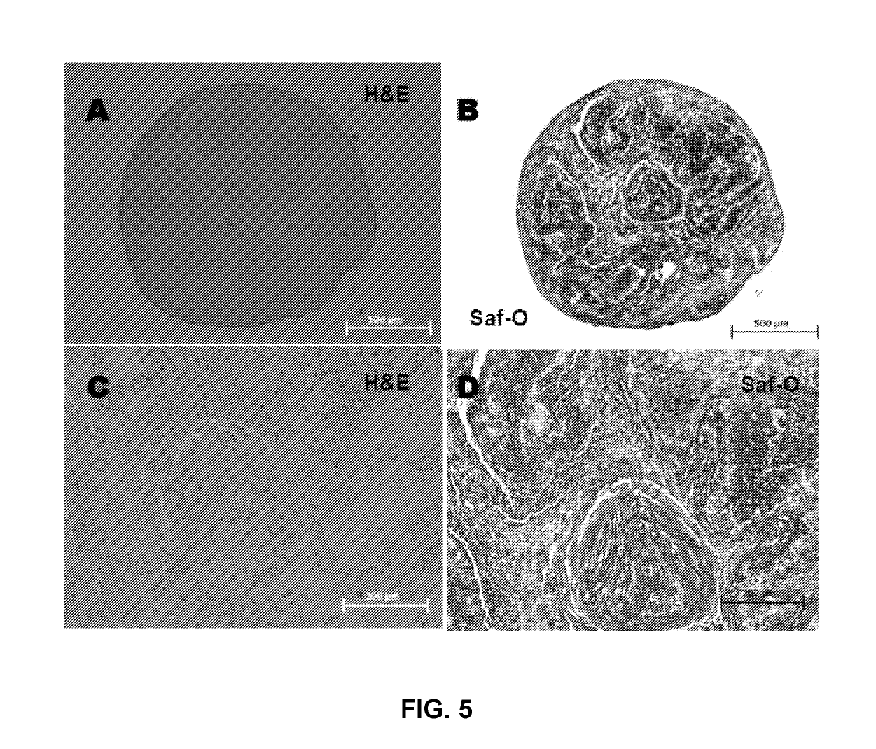

DSCs were differentiated into chondrocytes in DMEM supplemented with 10 ng/ml TGF-133. Safranin O straining and glycosaminoglycan (GAG) content assay were performed to evaluate chondrogenic differentiation (Blyscan™, Biocolor, UK) (FIG. 5).

### Example 2

### Insulin-Producing Cells (IPCs) from Dental-Pulp Stem/Progenitor Cells

This Example was presented as an abstract at the 2008 Tissue Engineering and Regenerative Medicine International Society (TERMIS) meeting, Dec. 7, 2008.

### Introduction.

Example 1 describes the isolation of DSCs from human deciduous and adult teeth, and their multi-lineage differentiation capacity into pancreatic beta-like cells, chondrocyte-like cells, and myocyte-like cells and hair follicle-like cells. The differentiation of polyclonal and monoclonal DSCs into insulin-producing cells (IPCs) is further described herein, using media differing from that used in Example 1. The DSC clones were differentiated into endoderm pancreatic cells and critical markers associated with IPC differentiation were characterized.

### Methods and Materials

Subjects and Cell Culture. Exfoliating deciduous incisors and permanent third molars of multiple donors were collected with IRB approval. The dental pulps were isolated and enzyme-digested. Mononucleated and adherent cells were cultured in DMEM-LG medium containing 10% FBS and 1% antibiotics in 10 cm cell culture dishes. Single cells in suspension were then isolated from heterogeneous DSCs and cultured under the same conditions for 2 weeks. Following this, the monoclonal cells were transferred to 6-well culture plates.

Differentiation of Insulin Producing Cells (IPCs). DSC clones were expanded and subjected to insulin-producing cell differentiation conditions. Briefly, 2.5×10⁵ DSCs were suspended in DMEM-LG medium containing 10% FBS and centrifuged for 5 min. Then, the cells were transferred into 1:1 DMEM/F-12 medium containing glucose, Insulin-Transferin-Selenium-A, IBMX, Wnt3a, and 5 μg/mL fibronectin, and subsequently cultured for 2 days. The cells were then switched to DMEM/F-12 medium containing glucose, nicotinamide, N2 supplement, B27 supplement, noggin (400 ng/ml), and fibronectin for 4 days. After suspension culture, the cell pellets were washed with PBS, fixed with 4% paraformaldehyde, and sectioned. The expression of proinsulin, insulin, and Pdx-1 were detected by immunofluorescence and ELISA.

### Results

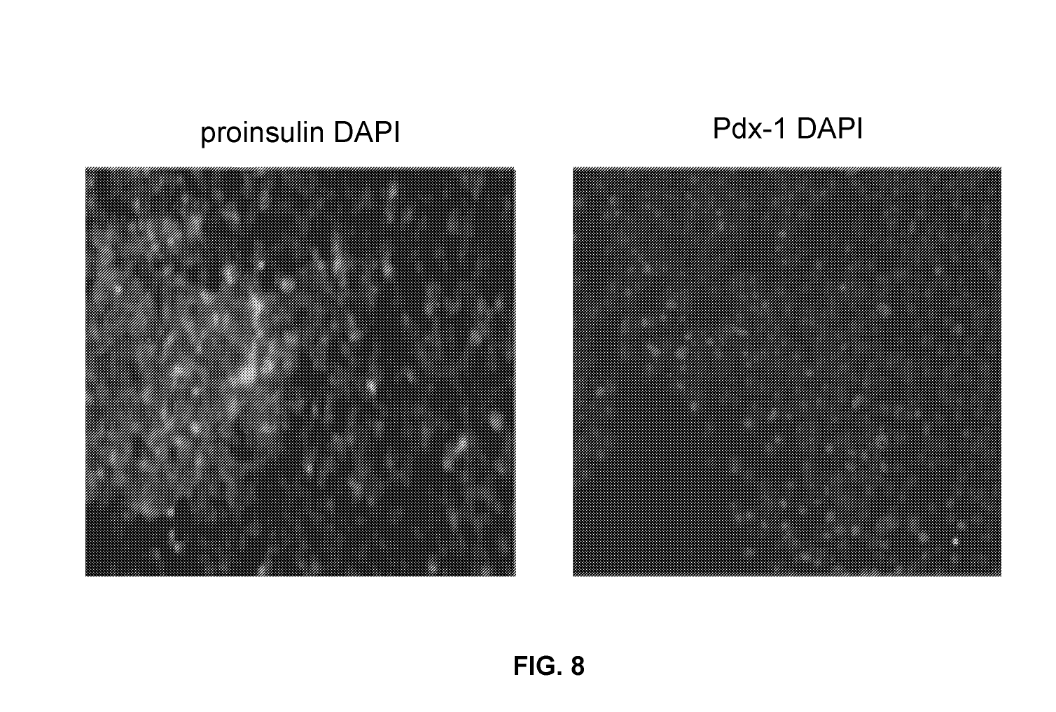

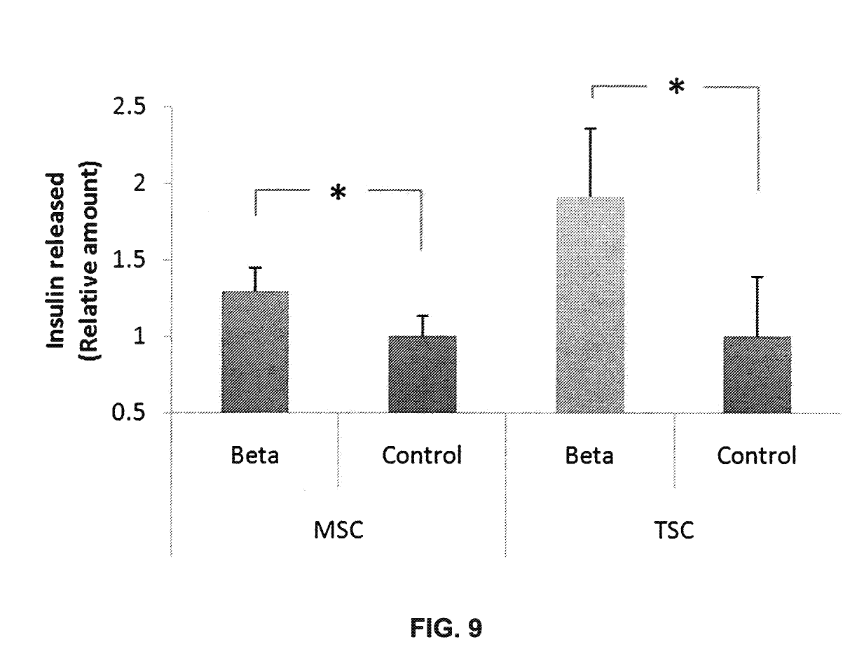

Insulin-producing cell differentiation. Overall, heterogeneous DSCs had a low yield of IPC differentiation. Differentiated heterogeneous DSCs expressed significantly more insulin, Pdx-1, and C-peptide, compared to the undifferentiated DSCs. Twenty clones isolated from 3 permanent teeth and 6 clones isolated from 2 deciduous teeth were used for IPC differentiation. Immunostaining demonstrated that 2 of 20 permanent teeth clones and 5 of 6 deciduous teeth clones were Stro-1 positive. Upon IPC differentiation, 2 permanent and 2 deciduous teeth clones demonstrated strong proinsulin and Pdx-1 staining (FIG. 8). Insulin production by heterogeneous IPCs was further validated by ELISA. Polyclonal DSCs produced twice the amount of insulin in comparison with bone marrow-derived MSCs (FIG. 9). At this time, the efficiency of cloned DSCs regarding proinsulin and Pdx-1 expression is markedly higher than that of polyclonal DSCs.

Cell marker analysis. Of the 4 DSC clones that were differentiated into IPCs, one permanent and one deciduous clone were Stro-1 positive, whereas the other two clones were Stro-1 negative. Whether Stro-1 is an accurate surrogate marker for IPCs warrants additional investigation. Interestingly, the strongest insulin-producing IPC clone was positive for both Nanog and Oct4, whereas the other 3 clones were positive for either Nanog or Oct4. These findings suggest that Nanog and/or Oct4, two hallmarks of embryonic stem cells, expressed by fractions of dental-pulp stem/progenitor cells, are indicative, but not obligatory, markers for IPCs differentiation.

### Discussion

This work demonstrates that insulin-producing cells can be derived from dental-pulp stem/progenitor cells, both polyclonal and monoclonal populations. Nanog and Oct4, two hallmarks expressed by embryonic stem cells, appear to be indicative, but not obligatory, markers for IPC differentiation. The insulin yield of polyclonal DSCs was approximately two fold higher than that of bone marrow-derived MSCs subjected to the same IPC differentiation protocol. It is anticipated that clonal DSCs have greater insulin yield than polyclonal DSCs, because cloned DSCs have higher differentiation efficiency towards IPCs than polyclonal DSCs. These discoveries offer a potential for utilizing dental-pulp stem/progenitor cells towards the derivation of insulin-producing cells. Advantages of IPC differentiation from dental-pulp stem/progenitor cells include: 1) DSCs are readily accessible from exfoliating/extracted teeth that are otherwise discarded as medical waste, 2) DSCs as postnatal stem cells are not subjected to ethical controversy, and 3) rapid proliferation of DSCs provide a potential for expansion.

### Example 3

### Culture of Dental Stem Cells

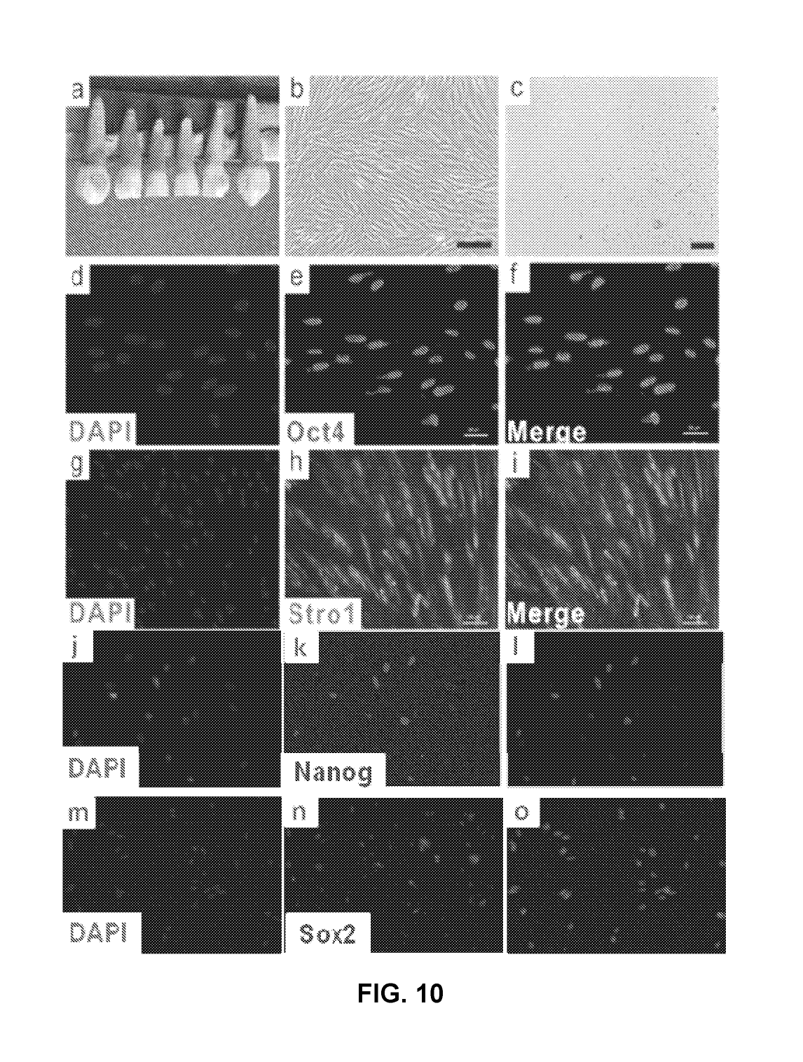

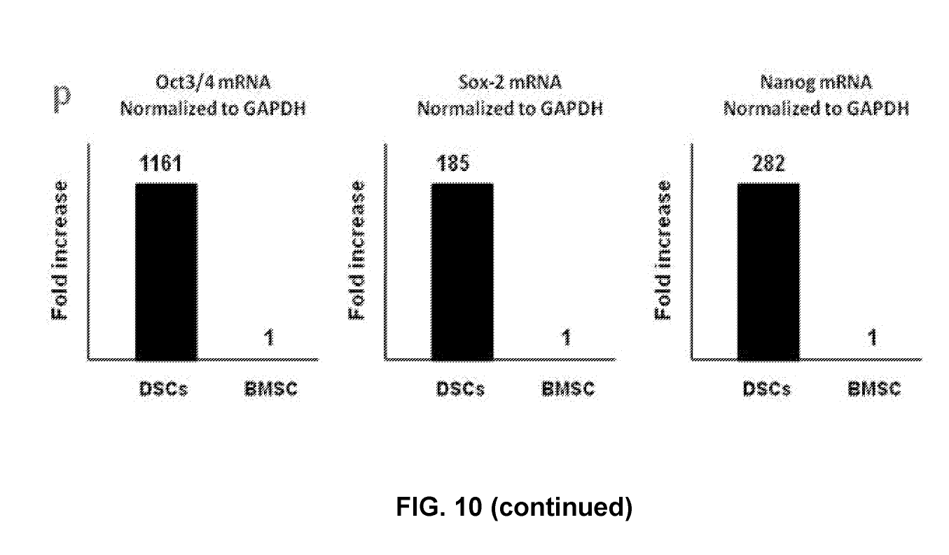

DSC isolation. Following IRB approval, deciduous teeth from multiple donors (FIG. 10a), were extracted in the dental clinics of Columbia University College of Dental Medicine at the time of exfoliation. The dental pulps were then digested with type I collagenase (2 mg/ml) and dispase (1 mg/ml) for 2 hr. The cells were plated into the type I collagen coated 10 cm cell culture dishes. Following medium change, mononucleated, adherent cells were re-plated (FIG. 10b). Single cell clones (FIG. 10c) were derived from the heterogeneous population (e.g., FIG. 10b), as described in Alhadlaq and Mao, 2004 and Marion and Mao, 2006. Two clonally expanded cell subpopulations are evident in FIG. 10c. Different clones behaved rather differently in population doubling time and differentiation capacity. Heterogeneous DSCs showed positive expression of Oct-3/4, Nanog and Sox2 (FIG. 10d-f, j-l, m-o), which are hallmarks of embryonic stem cells, as well as Stro1, a mesenchymal stem cell marker (FIG. 10g-i). DSCs display a heterogeneous character. At passage 1, about 30% of the cells were stro-1 positive, and 70% of the cells were positive for Nanog, Oct-3/4, and Sox2. The expression levels of the markers were examined by Taqman real-time RT-PCR. Compared to the bone marrow mesenchymal stem cells, the mRNA expression levels of Oct3/4, sox2 and nanog are 1161, 185 and 282 folds higher respectively (FIG. 10p).

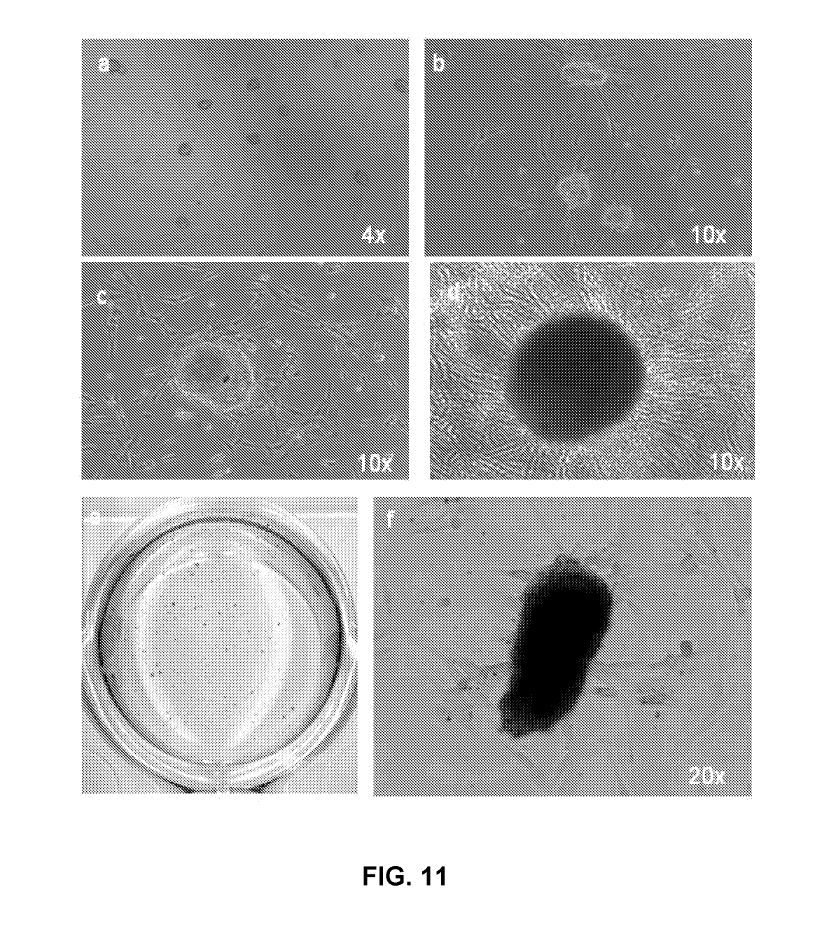

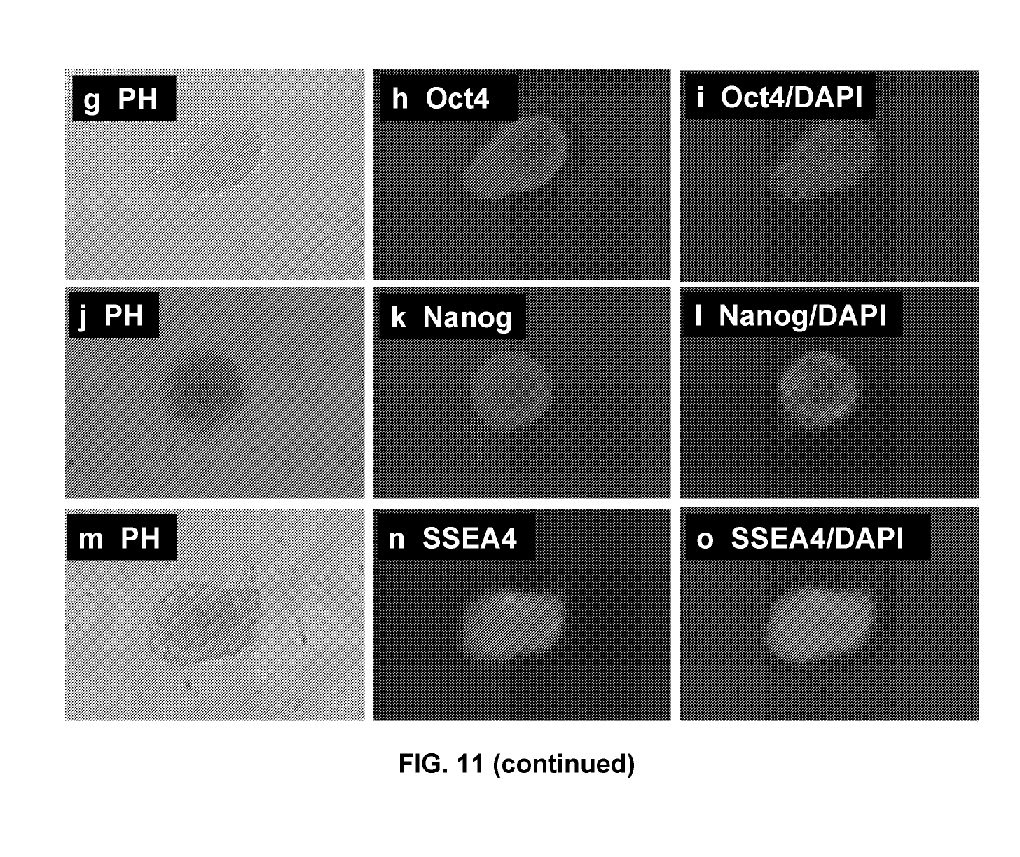

DSCs in embryonic stem cell medium. DSCs isolated from deciduous teeth were cultured in DMEM containing 10% FBS, 1% antibiotics for 2 weeks. Then the cells were transferred into ES cell culture medium (DMEM/F-12 containing 20% Knockout™ Serum Replacement, Leukemia Inhibitory Factor, 1% non-essential amino acids, 1% antibiotics and 0.1 mM β-mercaptoethanol). The cells form clusters in 2-5 days (FIG. 11a-c) and apparently, the fibroblast-like cells act as feeders which provide the adherent surface for the clusters. The DSCs' behavior in the presence of irradiated mouse embryonic fibroblast as feeders was also tested. In 2 weeks the cells formed bigger clusters up to 400 micrometer (FIG. 11d). The DSC clusters formed in ES cell medium were stained for alkaline phosphatase (ALP) activity according to the protocol described in Moioli et al., 2008. The clusters were ALP-positive (FIG. 11e-f), further indicating a ES-like state. The expression of the ES cell markers was also examined, including Oct-3/4, Nanog and SSEA4 by immunofluorescence staining as described in Takahashi and Yamanaka, 2006. The DSC clusters were Oct-3/4, Nanog and SSEA4-positive (FIG. 2g-o).

### REFERENCES

Alhadlaq A, Mao JJ 2004 Mesenchymal stem cells: isolation and therapeutics. Stem Cells Dev 13:436-448.
D'Amour K A et al. 2006 Production of pancreatic hormone-expressing endocrine cells from human embryonic stem cells. Nat Biotechnol 24:1392-1401.
Lumelsky N, Blondel O, Laeng P, Velasco I, Ravin R, McKay R 2001 Differentiation of embryonic stem cells to insulin-secreting structures similar to pancreatic islets. Science 292:1389-1394.
Mao J J et al. 2006 J Dent Res 85:966-979.
Marion N R, Mao J J 2006 Mesenchymal stem cells and tissue engineering. Methods Enzymol 420:339-361.
Miura M et al. 2003 Proc. Natl. Acad. Sci. USA 100:5807-5812.
Moioli E K, Clark P A, Chen M, Dennis J E, Erickson H P, Gerson S L, Mao J J 2008 Synergistic actions of hematopoietic and mesenchymal stem/progenitor cells in vascularizing bioengineered tissues. PLoS ONE 3:e3922.
Nakajima N et al. 2006 Biochem Biophys Res Comm 350:1006-1012.
Peptan I A et al. 2006 Plast Reconstr Surg 117:1462-1470.
Takahashi K, Yamanaka S 2006 Induction of pluripotent stem cells from mouse embryonic and adult fibroblast cultures by defined factors. Cell 126:663-676.
Timper et al. 2006 Biochem Biophys Res Comm 341:1135-1140.
Miura et al. 2003 PNAS100:5807-5812.
Morsczeck et al. 2005 Matrix Biol 24:155-165.
Reynolds et al. 2004 Differentiation 72:566-575.
Saito et al. 2006 Cancer Cell 9:435-443.
Yen and Sharpe 2007 Cell Tissue Res DOI 10.1007/s00441-007-0467-6 (review)
U.S. Pat. No. 6,767,740 B2
U.S. Pat. No. 7,052,907 B2
U.S. Pat. No. 7,326,572 B2
U.S. Patent Publication US070274958 A1
U.S. Patent Publication US080248005 A1
PCT Patent Publication WO0207679A2
PCT Patent Publication WO03066840 A2
PCT Patent Publication WO04073633 A2
PCT Patent Publication WO06010600A2
PCT Patent Publication WO07014639 A2

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1-37. (canceled)

38. A method of differentiating a dental stem cell comprising:
incubating a dental stem cell in a medium that induces the differentiation of a dental stem cell into an insulin-secreting cell under conditions such that the dental stem cell differentiates into the insulin-secreting cell or pancreatic beta-like cell;
incubating a dental stem cell in a medium that induces the differentiation of a dental stem cell into a chondrocyte-like cell under conditions such that the dental stem cell differentiates into the chondrocyte-like cell;
incubating a dental stem cell in a medium that induces the differentiation of a dental stem cell into a myocyte-like cell under conditions such that the dental stem cell differentiates into the myocyte-like cell; or
incubating a dental stem cell in a medium that induces the differentiation of a dental stem cell into a hair follicle-like cell under conditions such that the dental stem cell differentiates into the hair follicle-like cell.

39. The method of claim 38 comprising:
incubating a dental stem cell in a medium that induces the differentiation of a dental stem cell into an insulin-secreting cell under conditions such that the dental stem cell differentiates into the insulin-secreting cell or pancreatic beta-like cell;
wherein the medium comprises (i) activin, extendin, pentagastrin, and hepatocyte growth factor or (ii) noggin.

40. The method of claim 38 comprising:
incubating a dental stem cell in a medium that induces the differentiation of a dental stem cell into an insulin-secreting cell under conditions such that the dental stem cell differentiates into the insulin-secreting cell or pancreatic beta-like cell; and
testing the cell for a characteristic of a pancreatic beta cell.

41. The method of claim 40, wherein testing the cell for a characteristic of a pancreatic beta cell comprises testing the cell for one or more of secretion of insulin, PDX1, or C-peptide.

42. The method of claim 38 comprising:
incubating a dental stem cell in a medium that induces the differentiation of a dental stem cell into a chondrocyte-like cell under conditions such that the dental stem cell differentiates into the chondrocyte-like cell;
wherein the medium comprises TGF-β3.

43. The method of claim 38 comprising:
incubating a dental stem cell in a medium that induces the differentiation of a dental stem cell into a chondrocyte-like cell under conditions such that the dental stem cell differentiates into the chondrocyte-like cell; and
testing the cell for a characteristic of a chondrocyte.

44. The method of claim 43, wherein testing the cell for a characteristic of a chondrocyte comprises testing the cell for safranin O staining or glycosaminoglycan content.

45. The method of claim 38 comprising:
incubating a dental stem cell in a medium that induces the differentiation of a dental stem cell into a myocyte-like cell under conditions such that the dental stem cell differentiates into the myocyte-like cell;
wherein the medium comprises dexamethasone and hydrocortisone.

46. The method of claim 38 comprising:
incubating a dental stem cell in a medium that induces the differentiation of a dental stem cell into a myocyte-like cell under conditions such that the dental stem cell differentiates into the myocyte-like cell; and
testing the cell for a characteristic of a myocyte.

47. The method of claim 46, wherein testing the cell for a characteristic of a myocyte comprises testing the cell for myoD, myf5, desmin or myosin.

48. The method of claim 38 comprising:
incubating a dental stem cell in a medium that induces the differentiation of a dental stem cell into a hair follicle-like cell under conditions such that the dental stem cell differentiates into the hair follicle-like cell;
wherein the medium comprises dermal papilla media or outer root sheath media.

49. The method of claim 38 comprising:
incubating a dental stem cell in a medium that induces the differentiation of a dental stem cell into a hair follicle-like cell under conditions such that the dental stem cell differentiates into the hair follicle-like cell; and
testing the cell for a characteristic of a hair follicle.

50. The method of claim 49, wherein testing the cell for a characteristic of a hair follicle comprises testing the cell for CD44, Lef1, CD59 or CK14.

51. The method of claim 38, wherein the dental stem cell is a mammalian cell or a human cell.

52. The method of claim 38, comprising transfecting the cell with a nucleic acid encoding a functional polynucleotide or polypeptide that is expressed by the cell, wherein the nucleic acid encodes (i) a functional polypeptide comprising a growth factor; (ii) a functional polypeptide comprising a growth factor that promotes differentiation into an insulin-secreting cell; or (iii) a functional polynucleotide comprising an miRNA or an antisense RNA.

53. A composition comprising:
(i) (a) a dental stem cell and (b) an insulin-secreting cell or a pancreatic beta-like cell;
(ii) (a) a dental stem cell and (b) a chondrocyte-like cell, a myocyte-like cell, or a hair follicle-like cell;
(iii) a pancreatic beta-like cell differentiated from a dental stem cell; or
(iv) a chondrocyte-like cell, a myocyte-like cell, or a hair follicle-like cell, derived from a dental stem cell.

54. A method of preparing an embryonic stem cell-like cell comprising: culturing a dental stem cell in a medium that maintains an embryonic stem cell under conditions such that the dental stem cell dedifferentiates into the embryonic stem cell-like cell.

55. The method of claim 54, wherein the medium comprises:
(i) leukemia inhibitory factor (LIF) and Knockout™ Serum Replacement; or
(ii) feeder cells.

56. The method of claim 54, wherein the embryonic stem cell-like cell expresses alkaline phosphatase, Oct-3/4, Nanog and SSEA4.

57. The method of claim 54, comprising:
incubating the embryonic stem cell-like cell in a second medium that induces the differentiation of an embryonic stem cell-like cell into an insulin-secreting cell under conditions such that the embryonic stem cell-like cell differentiates into the insulin-secreting cell or pancreatic beta-like cell;
incubating the embryonic stem cell-like cell in a second medium that induces the differentiation of an embryonic stem cell-like cell into a chondrocyte-like cell under conditions such that the embryonic stem cell-like cell differentiates into the chondrocyte-like cell;
incubating the embryonic stem cell-like cell in a second medium that induces the differentiation of an embryonic stem cell-like cell into a myocyte-like cell under conditions such that the embryonic stem cell-like cell differentiates into the myocyte-like cell; or
incubating the embryonic stem cell-like cell in a second medium that induces the differentiation of an embryonic stem cell-like cell into a hair follicle-like cell under conditions such that the embryonic stem cell-like cell differentiates into the hair follicle-like cell.
